(19) United States
(12) Patent Application Publication
(10) Pub. No.: US 2020/0353022 A1
(43) Pub. Date: Nov. 12, 2020

(54) COMPOSITIONS AND METHODS FOR TREATING LIVER CANCER

(71) Applicants: MERCK SHARP & DOHME CORP., Rahway, NJ (US); AMGEN INC., Thousand Oaks, CA (US)

(72) Inventors: Jennifer Lorraine GANSERT, Simi Valley, CA (US); Swaminathan MURUGAPPAN, Thousand Oaks, CA (US); Michael Kevin WOLF, Thousand Oaks, CA (US)

(21) Appl. No.: 16/759,476

(22) PCT Filed: Oct. 26, 2018
(86) PCT No.: PCT/US2018/057731

§ 371 Date: Apr. 27, 2020

Related U.S. Application Data
(60) Provisional application No. 62/578,071, filed on Oct. 27, 2017.

(51) Int. Cl. A61K 35/763; A61K 39/395; A61P 35/00

(52) U.S. Cl. CPC A61K 35/763; A61K 2039/505; A61P 35/00; A61K 39/3955

(57) ABSTRACT

Methods and compositions for treating primary hepatic cancers and/or secondary hepatic cancers using a combination of talimogene laherparepvec and pembrolizumab, a pembrolizumab variant or an antigen-binding fragment thereof are provided.

### RELATED APPLICATION

This application claims the benefit of U.S. Provisional Application No. 62/578,071, filed on Oct. 27, 2017, which is incorporated herein by reference in its entirety.

### FIELD OF THE INVENTION

The present invention relates to the field of cancer therapeutics. In particular, the present invention relates to the treatment of primary or secondary hepatic cancer using a combination therapy comprising pembrolizumab, a pembrolizumab variant and/or an antigen-binding fragment thereof and talimogene laherparepvec.

### BACKGROUND

Primary liver cancer is globally the sixth most frequent cancer (6% of all cancers) and the second leading cause of death from cancer (9% of all cancer deaths) (World Cancer Report (2014) World Health Organization Chapters 1.1 and 5.6., ISBN 9283204298). In 2012, primary liver cancer affected 782,000 people, and 810,500 deaths were caused by primary liver cancer in 2015 (GBD 2015 Mortality and Causes of Death Collaborators, Lancet. 388 (10053): 1459-1544). The five-year survival rate for primary liver cancer is 18% in the United States (Cancer Stat Facts: Liver and Intrahepatic Bile Duct Cancer (URL: seer.cancer.gov/statfacts/html/livibd.html)).

Many cancers found in the liver are not true liver cancers, but are secondary liver cancers that arose from other sites in the body that have spread to the liver (i.e., metastases). Frequently, the site of origin is the gastrointestinal tract, since the liver is close to many of these metabolically active, blood-rich organs near to blood vessels and lymph nodes (such as pancreatic cancer, stomach cancer, colon cancer and carcinoid tumors mainly of the appendix). Secondary liver cancer may also derive from metastatic cancer of the breast, the ovary, the lung, the kidney and the prostate.

A clear need exists in the art for new methods and compositions for treating primary and secondary hepatic cancers.

### SUMMARY

The present disclosure is based in part on the discovery that combination therapy comprising pembrolizumab, a pembrolizumab variant and/or an antigen-binding fragment thereof and talimogene laherparepvec is useful in the treatment of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

The present disclosure is also based in part on the discovery that combination therapy comprising pembrolizumab, a pembrolizumab variant and/or an antigen-binding fragment thereof and talimogene laherparepvec is useful in the treatment of cancers such as primary and secondary hepatic cancers.

In one aspect, a method of treating a cancer in a subject, said method comprising administering to said subject talimogene laherparepvec, and pembrolizumab, a pembrolizumab variant or an antigen-binding fragment thereof, wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma, is provided.

In certain exemplary embodiments, talimogene laherparepvec is administered to the subject intratumorally, and/or pembrolizumab, a pembrolizumab variant or the antigen-binding fragment thereof is administered to the subject systemically. In other exemplary embodiments, talimogene laherparepvec is administered to the subject prior to or after the administration of pembrolizumab, a pembrolizumab variant or the antigen-binding fragment thereof. In other exemplary embodiments, talimogene laherparepvec is administered to the subject prior to the administration of pembrolizumab, a pembrolizumab variant or the antigen-binding fragment thereof.

In certain exemplary embodiments, talimogene laherparepvec is administered sequentially as an initial dose followed by one or more secondary doses. In other exemplary embodiments, pembrolizumab, a pembrolizumab variant or the antigen-binding fragment thereof is administered sequentially as an initial dose followed by one or more secondary doses. In still other exemplary embodiments, talimogene laherparepvec is administered sequentially as an initial dose followed by one or more secondary doses, and wherein pembrolizumab, a pembrolizumab variant or the antigen-binding fragment thereof is administered sequentially and concomitantly with one or more secondary doses of talimogene laherparepvec.

In certain exemplary embodiments, talimogene laherparepvec is administered intratumorally and wherein pembrolizumab, a pembrolizumab variant or the antigen-binding fragment thereof is administered systemically. In other exemplary embodiments, talimogene laherparepvec and pembrolizumab, a pembrolizumab variant or the antigen-binding fragment thereof are administered intratumorally.

In certain exemplary embodiments, a reduction in size of the injected tumor occurs after administering talimogene laherparepvec and pembrolizumab, a pembrolizumab variant or the antigen-binding fragment thereof.

In another aspect, a method of treating a primary or a secondary hepatic cancer in a subject comprising administering talimogene laherparepvec to the subject, and administering pembrolizumab, a pembrolizumab variant or an antigen-binding fragment thereof to the subject, is provided.

In certain exemplary embodiments, the primary hepatic cancer is a primary hepatocellular carcinoma, and/or the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally, and/or pembrolizumab, a pembrolizumab variant or the antigen-binding fragment thereof is administered to the subject systemically. In other exemplary embodiments, talimogene laherparepvec is administered to the subject prior to or after the administration of pembrolizumab, a pembrolizumab variant or the antigen-binding fragment thereof. In other exemplary embodiments, talimogene laherparepvec is administered to the subject prior to the administration of pembrolizumab, a pembrolizumab variant or the antigen-binding fragment thereof.

In certain exemplary embodiments, talimogene laherparepvec is administered sequentially as an initial dose followed by one or more secondary doses. In other exemplary embodiments, pembrolizumab, a pembrolizumab variant or the antigen-binding fragment thereof is administered sequentially as an initial dose followed by one or more secondary doses. In still other exemplary embodiments, talimogene laherparepvec is administered sequentially as an initial dose followed by one or more secondary doses, and wherein pembrolizumab, a pembrolizumab variant or the antigen-binding fragment thereof is administered sequentially and concomitantly with one or more secondary doses of talimogene laherparepvec.

In certain exemplary embodiments, talimogene laherparepvec is administered intratumorally and wherein pembrolizumab, a pembrolizumab variant or the antigen-binding fragment thereof is administered systemically. In other exemplary embodiments, talimogene laherparepvec and pembrolizumab, a pembrolizumab variant or the antigen-binding fragment thereof are administered intratumorally.

In certain exemplary embodiments, a reduction in size of the injected tumor occurs after administering talimogene laherparepvec and pembrolizumab, a pembrolizumab variant or the antigen-binding fragment thereof.

In another aspect, a method of treating a cancer in a subject that is poorly responsive to standard of care systemic anti-cancer therapy, said method comprising administering to said subject talimogene laherparepvec, and pembrolizumab, a pembrolizumab variant or an antigen-binding fragment thereof, wherein the standard of care systemic anti-cancer therapy does not comprise talimogene laherparepvec/pembrolizumab combination therapy, and wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma, is provided.

In certain exemplary embodiments, talimogene laherparepvec is administered to the subject intratumorally and/or the pembrolizumab variant, or the antigen-binding fragment thereof is administered to the subject systemically.

In another aspect, a method of treating a primary or a secondary hepatic cancer in a subject that is poorly responsive to standard of care systemic anti-cancer therapy, said method comprising administering to said subject talimogene laherparepvec, and pembrolizumab, a pembrolizumab variant or an antigen-binding fragment thereof, wherein the standard of care systemic anti-cancer therapy does not comprise talimogene laherparepvec/pembrolizumab combination therapy, is provided.

In certain exemplary embodiments, the primary hepatic cancer is a primary hepatocellular carcinoma, or the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally and/or the pembrolizumab variant, or the antigen-binding fragment thereof is administered to the subject systemically.

In another aspect, a method of treating a cancer in a subject that progressed during standard of care systemic anti-cancer therapy, said method comprising administering to said subject talimogene laherparepvec and pembrolizumab, a pembrolizumab variant or an antigen-binding fragment thereof, wherein the standard of care systemic anti-cancer therapy does not comprise talimogene laherparepvec/pembrolizumab combination therapy, and wherein said cancer is selected from the group consisting of: hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma, is provided.

In certain exemplary embodiments, talimogene laherparepvec is administered to the subject intratumorally and/or pembrolizumab, the pembrolizumab variant, or the antigen-binding fragment thereof is administered to the subject systemically.

In another aspect, a method of treating a primary or a secondary hepatic cancer in a subject that progressed during standard of care systemic anti-cancer therapy, said method comprising administering to said subject talimogene laherparepvec, and pembrolizumab, a pembrolizumab variant or an antigen-binding fragment thereof, wherein the standard of care systemic anti-cancer therapy does not comprise talimogene laherparepvec/pembrolizumab combination therapy, is provided.

In certain exemplary embodiments, the primary hepatic cancer is a primary hepatocellular carcinoma, or the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally and/or pembrolizumab, the pembrolizumab variant, or the antigen-binding fragment thereof is administered to the subject systemically.

In another aspect, a method of treating a cancer in a subject that is resistant to standard of care systemic anti-cancer therapy, said method comprising administering to said subject talimogene laherparepvec, and pembrolizumab, a pembrolizumab variant or an antigen-binding fragment thereof, wherein standard of care systemic anti-cancer therapy does not comprise talimogene laherparepvec/pembrolizumab combination therapy, and wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma, is provided.

In certain exemplary embodiments, talimogene laherparepvec is administered to the subject intratumorally and/or pembrolizumab, the pembrolizumab variant, or the antigen-binding fragment thereof is administered to the subject systemically.

In another aspect, a method of treating a primary or a secondary hepatic cancer in a subject that is resistant to standard of care systemic anti-cancer therapy, said method comprising administering to said subject talimogene laherparepvec, and pembrolizumab, a pembrolizumab variant or an antigen-binding fragment thereof, wherein standard of care systemic anti-cancer therapy does not comprise talimogene laherparepvec/pembrolizumab combination therapy, is provided.

In certain exemplary embodiments, the primary hepatic cancer is a primary hepatocellular carcinoma, or the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally and/or pembrolizumab, the pembrolizumab variant, or the antigen-binding fragment thereof is administered to the subject systemically.

In another aspect, a method of treating a cancer in a subject, said method comprising administering to said subject talimogene laherparepvec intratumorally as an initial dose followed by one or more secondary doses, and pembrolizumab, a pembrolizumab variant or an antigen-binding fragment thereof systemically as an initial dose followed by one or more secondary doses, is provided.

In certain exemplary embodiments, the secondary doses are administered every three weeks (Q3W). In certain exemplary embodiments, the initial dose of talimogene laherparepvec is administered on day 1 of week 1 and a secondary dose of talimogene laherparepvec is administered on day 1 of week 4, on day 1 of week 7, and Q3W thereafter. In certain exemplary embodiments, the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3W thereafter.

In certain exemplary embodiments, the initial dose of talimogene laherparepvec is administered at a dose of 10⁶ plaque forming units (PFU)/mL and the secondary doses of talimogene laherparepvec are administered at a dose of 10⁷ or 10⁸ PFU/mL.

In certain exemplary embodiments, the initial dose and the secondary doses are up to about 4 mL or about 8 mL. In certain exemplary embodiments, the initial dose and/or the secondary doses are each up to about 4 mL. In certain exemplary embodiments, the initial dose and/or the secondary doses are each up to about 8 mL.

In certain exemplary embodiments, the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

In another aspect, a method of treating a primary or a secondary hepatic cancer in a subject, said method comprising administering to said subject talimogene laherparepvec intratumorally as an initial dose followed by one or more secondary doses, and pembrolizumab, a pembrolizumab variant or an antigen-binding fragment thereof systemically as an initial dose followed by one or more secondary doses, is provided.

In certain exemplary embodiments, the secondary doses are administered every three weeks (Q3W). In certain exemplary embodiments, the initial dose of talimogene laherparepvec is administered on day 1 of week 1 and a secondary dose of talimogene laherparepvec is administered on day 1 of week 4, on day 1 of week 7, and Q3W thereafter. In certain exemplary embodiments, the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3W thereafter.

In certain exemplary embodiments, the initial dose of talimogene laherparepvec is administered at a dose of 10⁶ plaque forming units (PFU)/mL and the secondary doses of talimogene laherparepvec are administered at a dose of 10⁷ or 10⁸ PFU/mL.

In certain exemplary embodiments, the initial dose and the secondary doses are up to about 4 mL or about 8 mL. In certain exemplary embodiments, the initial dose and/or the secondary doses are each up to about 4 mL. In certain exemplary embodiments, the initial dose and/or the secondary doses are each up to about 8 mL.

In certain exemplary embodiments, the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

In another aspect, talimogene laherparepvec is provided for use in treating a cancer in a subject in combination with pembrolizumab, a pembrolizumab variant or an antigen-binding fragment thereof, wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally. In certain exemplary embodiments, pembrolizumab, the pembrolizumab variant or the antigen-binding fragment thereof is administered to the subject systemically. In certain exemplary embodiments, talimogene laherparepvec is administered to the subject prior or after to the administration of pembrolizumab, the pembrolizumab variant or the antigen-binding fragment thereof. In certain exemplary embodiments, talimogene laherparepvec is administered to the subject prior to the administration of pembrolizumab, the pembrolizumab variant or the antigen-binding fragment thereof.

In certain exemplary embodiments, a reduction in size of the injected tumor occurs after administering talimogene laherparepvec and pembrolizumab, the pembrolizumab variant or the antigen-binding fragment thereof.

In certain exemplary embodiments, talimogene laherparepvec is administered sequentially as an initial dose followed by one or more secondary doses. In certain exemplary embodiments, pembrolizumab, the pembrolizumab variant or the antigen-binding fragment thereof is administered sequentially as an initial dose followed by one or more secondary doses. In certain exemplary embodiments, talimogene laherparepvec is administered sequentially as an initial dose followed by one or more secondary doses, and wherein pembrolizumab, the pembrolizumab variant or the antigen-binding fragment thereof is administered sequentially and concomitantly with one or more secondary doses of talimogene laherparepvec. In certain exemplary embodiments, talimogene laherparepvec is administered intratumorally and wherein pembrolizumab, the pembrolizumab variant or the antigen-binding fragment thereof is administered systemically. In certain exemplary embodiments, talimogene laherparepvec and pembrolizumab, the pembrolizumab variant or the antigen-binding fragment thereof are administered intratumorally.

In certain exemplary embodiments, the secondary doses are administered Q3W. In certain exemplary embodiments, the initial dose of talimogene laherparepvec is administered on day 1 of week 1 and a secondary dose of talimogene laherparepvec is administered on day 1 of week 4, on day 1 of week 7, and Q3W thereafter. In certain exemplary embodiments, the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3W thereafter.

In certain exemplary embodiments, the initial dose of talimogene laherparepvec is administered at a dose of 10⁶ PFU/mL and the secondary doses of talimogene laherparepvec are administered at a dose of 10⁷ or 10⁸ PFU/mL. In certain exemplary embodiments, the initial dose and the secondary doses are up to about 4 mL or about 8 mL. In certain exemplary embodiments, the initial dose and/or the secondary doses are each up to about 4 mL. In certain exemplary embodiments, the initial dose and/or the secondary doses are each up to about 8 mL. In certain exemplary embodiments, the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

In another aspect, pembrolizumab, a pembrolizumab variant or an antigen-binding fragment thereof is provided for use in treating a cancer in a subject in combination with talimogene laherparepvec, wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally. In certain exemplary embodiments, pembrolizumab, the pembrolizumab variant or the antigen-binding fragment thereof is administered to the subject systemically. In certain exemplary embodiments, talimogene laherparepvec is administered to the subject prior or after to the administration of pembrolizumab, the pembrolizumab variant or the antigen-binding fragment thereof. In certain exemplary embodiments, talimogene laherparepvec is administered to the subject prior to the administration of pembrolizumab, the pembrolizumab variant or the antigen-binding fragment thereof.

In certain exemplary embodiments, a reduction in size of the injected tumor occurs after administering talimogene laherparepvec and pembrolizumab, the pembrolizumab variant or the antigen-binding fragment thereof.

In certain exemplary embodiments, talimogene laherparepvec is administered sequentially as an initial dose followed by one or more secondary doses. In certain exemplary embodiments, pembrolizumab, the pembrolizumab variant or the antigen-binding fragment thereof is administered sequentially as an initial dose followed by one or more secondary doses. In certain exemplary embodiments, talimogene laherparepvec is administered sequentially as an initial dose followed by one or more secondary doses, and wherein pembrolizumab, the pembrolizumab variant or the antigen-binding fragment thereof is administered sequentially and concomitantly with one or more secondary doses of talimogene laherparepvec. In certain exemplary embodiments, talimogene laherparepvec is administered intratumorally and wherein pembrolizumab, the pembrolizumab variant or the antigen-binding fragment thereof is administered systemically. In certain exemplary embodiments, talimogene laherparepvec and pembrolizumab, the pembrolizumab variant or the antigen-binding fragment thereof are administered intratumorally.

In certain exemplary embodiments, the secondary doses are administered Q3W. In certain exemplary embodiments, the initial dose of talimogene laherparepvec is administered on day 1 of week 1 and a secondary dose of talimogene laherparepvec is administered on day 1 of week 4, on day 1 of week 7, and Q3W thereafter. In certain exemplary embodiments, the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3W thereafter.

In certain exemplary embodiments, the initial dose of talimogene laherparepvec is administered at a dose of 10⁶ PFU/mL and the secondary doses of talimogene laherparepvec are administered at a dose of 10⁷ or 10⁸ PFU/mL. In certain exemplary embodiments, the initial dose and the secondary doses are up to about 4 mL or about 8 mL. In certain exemplary embodiments, the initial dose and/or the secondary doses are each up to about 4 mL. In certain exemplary embodiments, the initial dose and/or the secondary doses are each up to about 8 mL. In certain exemplary embodiments, the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

In another aspect, talimogene laherparepvec is provided for use in treating a primary or a secondary hepatic cancer in a subject in combination with pembrolizumab, a pembrolizumab variant or an antigen-binding fragment thereof.

In certain exemplary embodiments, the primary hepatic cancer is an HCC or the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally. In certain exemplary embodiments, pembrolizumab, the pembrolizumab variant or the antigen-binding fragment thereof is administered to the subject systemically. In certain exemplary embodiments, talimogene laherparepvec is administered to the subject prior to or after the administration of pembrolizumab, the pembrolizumab variant or the antigen-binding fragment thereof. In certain exemplary embodiments, talimogene laherparepvec is administered to the subject prior to the administration of pembrolizumab, the pembrolizumab variant or the antigen-binding fragment thereof.

In certain exemplary embodiments, a reduction in size of the injected tumor occurs after administering talimogene laherparepvec and pembrolizumab, the pembrolizumab variant or the antigen-binding fragment thereof.

In certain exemplary embodiments, talimogene laherparepvec is administered sequentially as an initial dose followed by one or more secondary doses. In certain exemplary embodiments, pembrolizumab, the pembrolizumab variant or the antigen-binding fragment thereof is administered sequentially as an initial dose followed by one or more secondary doses. In certain exemplary embodiments, talimogene laherparepvec is administered sequentially as an initial dose followed by one or more secondary doses, and wherein pembrolizumab, the pembrolizumab variant or the antigen-binding fragment thereof is administered sequentially and concomitantly with one or more secondary doses of talimogene laherparepvec. In certain exemplary embodiments, talimogene laherparepvec is administered intratumorally and wherein pembrolizumab, the pembrolizumab variant or the antigen-binding fragment thereof is administered systemically. In certain exemplary embodiments, talimogene laherparepvec and pembrolizumab, the pembrolizumab variant or the antigen-binding fragment thereof are administered intratumorally.

In certain exemplary embodiments, the secondary doses are administered Q3W. In certain exemplary embodiments, the initial dose of talimogene laherparepvec is administered on day 1 of week 1 and a secondary dose of talimogene laherparepvec is administered on day 1 of week 4, on day 1 of week 7, and Q3W thereafter. In certain exemplary embodiments, the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3 W thereafter.

In certain exemplary embodiments, the initial dose of talimogene laherparepvec is administered at a dose of 10⁶ PFU/mL and the secondary doses of talimogene laherparepvec are administered at a dose of 10⁷ or 10⁸ PFU/mL. In certain exemplary embodiments, the initial dose and the secondary doses are up to about 4 mL or about 8 mL. In certain exemplary embodiments, the initial dose and/or the secondary doses are each up to about 4 mL. In certain exemplary embodiments, the initial dose and/or the secondary doses are each up to about 8 mL. In certain exemplary embodiments, the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

In another aspect, pembrolizumab, a pembrolizumab variant or an antigen-binding fragment thereof is provided for use in treating a primary or a secondary hepatic cancer in a subject in combination with talimogene laherparepvec.

In certain exemplary embodiments, the primary hepatic cancer is an HCC or the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In certain exemplary embodiments, talimogene laherparepvec is administered to the subject intratumorally. In certain exemplary embodiments, pembrolizumab, the pembrolizumab variant or the antigen-binding fragment thereof is administered to the subject systemically. In certain exemplary embodiments, talimogene laherparepvec is administered to the subject prior to or after the administration of pembrolizumab, the pembrolizumab variant or the antigen-binding fragment thereof. In certain exemplary embodiments, talimogene laherparepvec is administered to the subject prior to the administration of pembrolizumab, the pembrolizumab variant or the antigen-binding fragment thereof.

In certain exemplary embodiments, a reduction in size of the injected tumor occurs after administering talimogene laherparepvec and pembrolizumab, the pembrolizumab variant or the antigen-binding fragment thereof.

In certain exemplary embodiments, talimogene laherparepvec is administered sequentially as an initial dose followed by one or more secondary doses. In certain exemplary embodiments, pembrolizumab, the pembrolizumab variant or the antigen-binding fragment thereof is administered sequentially as an initial dose followed by one or more secondary doses. In certain exemplary embodiments, talimogene laherparepvec is administered sequentially as an initial dose followed by one or more secondary doses, and wherein pembrolizumab, the pembrolizumab variant or the antigen-binding fragment thereof is administered sequentially and concomitantly with one or more secondary doses of talimogene laherparepvec. In certain exemplary embodiments, talimogene laherparepvec is administered intratumorally and wherein pembrolizumab, the pembrolizumab variant or the antigen-binding fragment thereof is administered systemically. In certain exemplary embodiments, talimogene laherparepvec and pembrolizumab, the pembrolizumab variant or the antigen-binding fragment thereof are administered intratumorally.

In certain exemplary embodiments, the secondary doses are administered Q3W. In certain exemplary embodiments, the initial dose of talimogene laherparepvec is administered on day 1 of week 1 and a secondary dose of talimogene laherparepvec is administered on day 1 of week 4, on day 1 of week 7, and Q3W thereafter. In certain exemplary embodiments, the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3 W thereafter.

In certain exemplary embodiments, the initial dose of talimogene laherparepvec is administered at a dose of 10⁶ PFU/mL and the secondary doses of talimogene laherparepvec are administered at a dose of 10⁷ or 10⁸ PFU/mL. In certain exemplary embodiments, the initial dose and the secondary doses are up to about 4 mL or about 8 mL. In certain exemplary embodiments, the initial dose and/or the secondary doses are each up to about 4 mL. In certain exemplary embodiments, the initial dose and/or the secondary doses are each up to about 8 mL. In certain exemplary embodiments, the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

The summary of the disclosure described above is non-limiting and other features and advantages of the disclosed biomarkers and methods will be apparent from the following drawings, the detailed description of the disclosure, the example and the claims.

### BRIEF DESCRIPTION OF THE DRAWINGS

FIG. 1 depicts study design and treatment schema for Part 1, Group A, showing talimogene laherparepvec monotherapy cohorts and talimogene laherparepvec plus pembrolizumab combination therapy cohorts. DLT=dose-limiting toxicity; HCC=hepatocellular carcinoma; MTC=maximum tolerated concentration; MTV=maximum tolerated volume; PFU=plaque forming unit; T-VEC=talimogene laherparepvec. ^{a}First dose concentration of talimogene laherparepvec is always 10⁶ PFU/mL. ^{b}Cohort 4 will be opened only if one of these conditions are met: 1) DLT≥33% in Cohort 2, or 2) DLT≥33% in cohort 3 and Part 2 dose for talimogene laherparepvec not determined yet, or 3) DLT≥33% in cohort 3 and Part 2 concentration for talimogene laherparepvec is determined to be 10⁷ PFU/mL. ^{c}MTV determined from monotherapy cohorts, when available, may be used in Part 2. ^{d}If both cohorts 3 or 4 and the combination cohorts (5 or 6) are open in the same institution, for subjects with a tumor burden who can receive 8 mL, enrollment into Cohort 3 or 4 must be strongly preferred until the MTV in monotherapy is determined. ^{e}Cohort 1 of Group B will be initiated only after safety has been established in Cohort 1 of Group A.

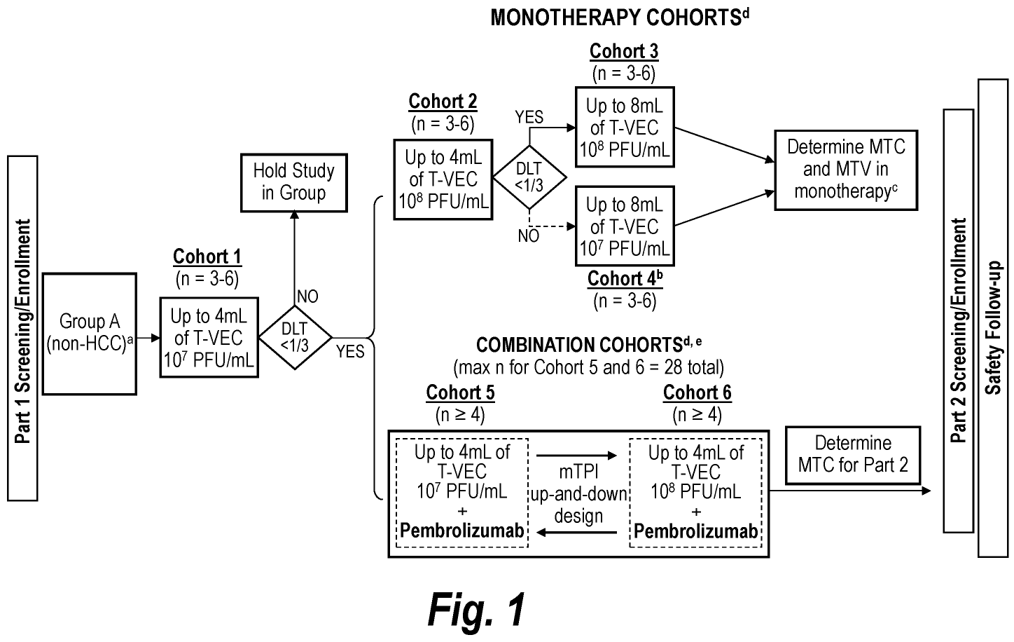

FIG. 2 depicts study design and treatment schema for Part 1, Group B, showing talimogene laherparepvec monotherapy cohorts and talimogene laherparepvec plus pembrolizumab combination therapy cohorts. DLT=dose-limiting toxicity; HCC=hepatocellular carcinoma; MTC=maximum tolerated concentration; MTV=maximum tolerated volume; PFU=plaque forming unit; T-VEC=talimogene laherparepvec. ^{a}First dose concentration of talimogene laherparepvec is always 10⁶ PFU/mL. ^{b}Cohort 4 will be opened only if one of these conditions are met: 1) DLT≥33% in Cohort 2, or 2) DLT≥33% in cohort 3 and Part 2 dose for talimogene laherparepvec not determined yet, or 3) DLT≥33% in cohort 3 and Part 2 concentration for talimogene laherparepvec is determined to be 10⁷ PFU/mL. ^{c}MTV determined from monotherapy cohorts, when available, may be used in Part 2. ^{d}If both cohorts 3 or 4 and the combination cohorts (5 or 6) are open in the same institution, for subjects with a tumor burden who can receive 8 mL, enrollment into Cohort 3 or 4 must be strongly preferred until the MTV in monotherapy is determined. ^{e}Cohort 1 of Group B will be initiated only after safety has been established in Cohort 1 of Group A.

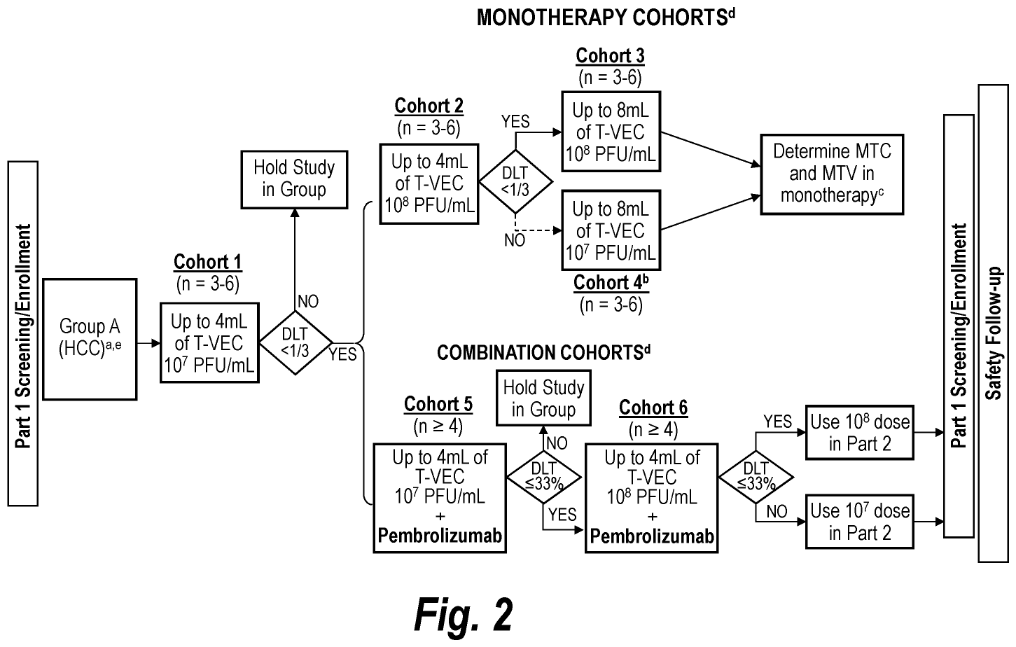

FIG. 3 depicts study design and treatment schema for Part 2 to assess efficacy of combination therapy in seven tumor types: primary hepatocellular carcinoma (HCC); breast adenocarcinoma (BC) with liver metastases; colorectal adenocarcinoma (CRC) with liver metastases; gastroesophageal cancer (GEC) (adenocarcinoma or squamous cell carcinoma) with liver metastases; melanoma with liver metastases; non-small cell lung cancer (NSCLC) with liver metastases; and clear cell renal cell carcinoma (RCC) with liver metastases. MTC=maximum tolerated concentration; MTV=maximum tolerated volume; NSCLC=non-small cell lung cancer; RCC=clear cell renal cell carcinoma; T-VEC=talimogene laherparepvec.

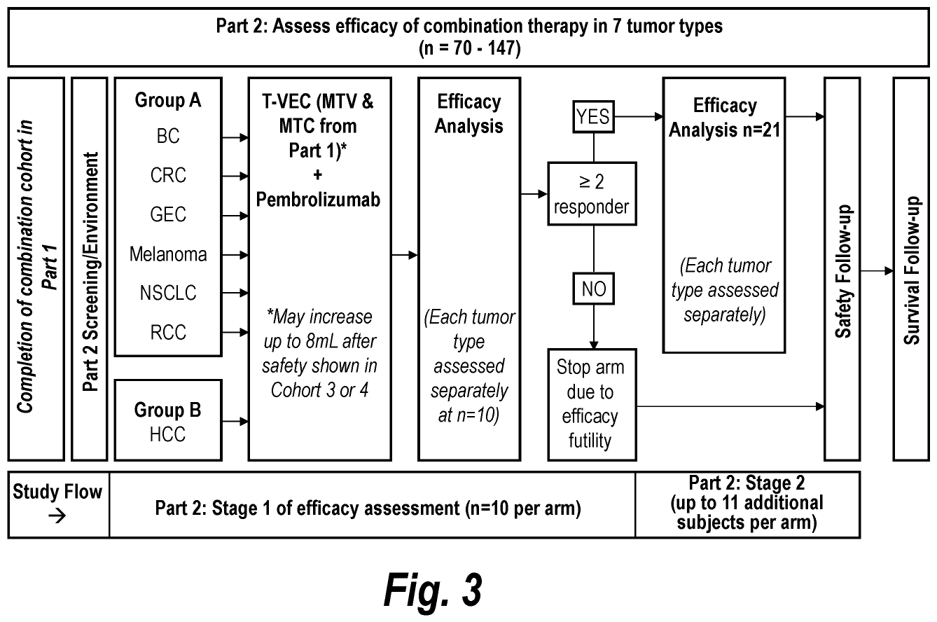

### DETAILED DESCRIPTION OF EXEMPLARY EMBODIMENTS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the dosage of pembrolizumab, a pembrolizumab variant and/or antigen-binding fragment thereof or talimogene laherparepvec, or the length of treatment time with pembrolizumab, a pembrolizumab variant and/or antigen-binding fragment thereof or talimogene laherparepvec) means that the parameter may vary by 1%, 2%, 3%, 4%, 5%, 6%, 7%, 8%, 9% or 10% above or below the stated numerical value for that parameter.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

As used herein, the term “antibody” refers to any form of antibody that exhibits the desired biological or binding activity. Thus, it is used in the broadest sense and specifically covers, but is not limited to, monoclonal antibodies (including full-length monoclonal antibodies), polyclonal antibodies, multi-specific antibodies (e.g., bispecific antibodies), humanized antibodies, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)).

The variable regions of each light/heavy chain pair form the antibody binding site. Thus, in general, an intact antibody has two binding sites. Except in bifunctional or bispecific antibodies, the two binding sites are, in general, the same.

“Variable regions” or “V region” as used herein means the segment of IgG chains which is variable in sequence between different antibodies. It extends to Kabat residue 109 in the light chain and 113 in the heavy chain.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia et al., (1987) J Mol. Biol. 196:901-917 or Chothia et al., (1989) Nature 342:878-883.

As used herein, the term “hypervariable region” refers to the amino acid residues of an antibody that are responsible for antigen-binding. The hypervariable region comprises amino acid residues from a CDR (i.e. LCDR1, LCDR2 and LCDR3 in the light chain variable domain and HCDR1, HCDR2 and HCDR3 in the heavy chain variable domain) See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure).

As used herein, unless otherwise indicated, “antibody fragment” or “antigen-binding fragment” refers to antigen-binding fragments of antibodies, i.e., antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments; diabodies; linear antibodies; single-chain antibody molecules, e.g., sc-Fv; nanobodies and multi-specific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two-fold greater, preferably at least ten times greater, more preferably at least 20 times greater, and most preferably at least 100 times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Biotherapeutic agent” means a biological molecule, such as an antibody, that blocks ligand/receptor signaling in any biological pathway that supports tumor maintenance and/or growth or suppresses the anti-tumor immune response.

The terms “cancer,” “cancerous,” or “malignant” refer to or describe the physiological condition in mammals that is typically characterized by unregulated cell growth. Examples of particular hepatic cancers that are responsive to talimogene laherparepvec/pembrolizumab combination therapy are primary hepatic cancers and secondary hepatic cancers caused by metastasis of a non-primary hepatic cancer.

Primary hepatic cancers include, but are not limited to, hepatocellular carcinoma (HCC), bile duct cancer, fibrolamellar HCC, hemangiosarcoma, angiosarcoma and hepatoblastoma. In a particular embodiment, a primary hepatic tumor is HCC.

Secondary hepatic cancers are caused by a metastasis of one or more cancer types that include, but are not limited to, hepatocellular carcinoma, breast cancer (e.g., endocrine receptor positive (ER+) breast cancer, HER2 positive (HER2+) breast cancer, triple-negative breast cancer, triple positive breast cancer and the like), colon cancer, colorectal cancer, kidney cancer, esophageal cancer, lung cancer (e.g., non-small cell lung cancer, small cell lung cancer), melanoma (including uveal melanoma), ovarian cancer, uterine cancer, pancreatic cancer and stomach cancer. In a particular embodiment, a secondary hepatic tumor is caused by a metastasis of hepatocellular carcinoma, breast adenocarcinoma (BC), colorectal adenocarcinoma (CRC), gastroesophageal (GEC) adenocarcinoma, GEC squamous cell carcinoma (SCC), melanoma (including uveal melanoma), non-small cell lung cancer (NSCLC), or clear cell renal cell carcinoma (RCC).

“CDR” or “CDRs” as used herein means complementarity determining region(s) in an immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et al., JMB 273:927-948 (1997), incorporated by reference herein.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering (or substantially altering) the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

“Comprising” or variations such as “comprise,” “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, if a gene signature score is defined as the composite RNA expression score for a set of genes that consists of a specified list of genes, the skilled artisan will understand that this gene signature score could include the RNA expression level determined for one or more additional genes, preferably no more than three additional genes, if such inclusion does not materially affect the predictive power.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYS LEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab,” as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Interferon gamma” and “IFNγ” (also called immune or type II interferon), refers to a pleiotropic cytokine involved in the regulation of nearly all phases of immune and inflammatory responses, including the activation, growth and differentiation of T-cells, B-cells, macrophages, NK cells and other cell types such as endothelial cells and fibroblasts. IFNγ enhances MHC expression on antigen-presenting cells, and also plays an important role in activating lymphocytes to enhance anti-tumor effects.

IFNγ can contribute to the containment of tumor progression and growth by increasing tumor antigen presentation to tumor-specific T-cells and increasing susceptibility to NK cytotoxicity. In addition to promoting an immune response to the tumor, IFN-γ can also induce expression of tumor suppressing factors.

“Genetically modified oncolytic virus,” as used herein, refers to an oncolytic virus that has been modified as compared to a wild-type version of the virus, typically to remove and/or insert one or more genes. A preferred genetically modified oncolytic virus of the invention is talimogene laherparepvec, also known as IMLYGIC® (INN=talimogene laherparepvec), a genetically engineered herpes virus that is commercially available from Amgen Inc. (Thousand Oaks, Calif.). Talimogene laherparepvec is described in, e.g., WO 2014036412, incorporated herein by reference in its entirety for all purposes.

Talimogene laherparepvec, HSV-1 (strain JS1) ICP34.5-/ICP47-/hGM-CSF (previously known as OncoVex^{GM-CSF}), is an intratumorally delivered oncolytic immunotherapy comprising an immune-enhanced HSV-1 that selectively replicates in solid tumors. (Lui et al., Gene Therapy, 10:292-303, 2003; U.S. Pat. Nos. 7,223,593 and 7,537,924.) The HSV-1 was derived from Strain JS1 as deposited at the European collection of cell cultures (ECAAC) under accession number 01010209. In talimogene laherparepvec, the HSV-1 viral genes encoding ICP34.5 have been functionally deleted. Functional deletion of ICP34.5, which acts as a virulence factor during HSV infection, limits replication in non-dividing cells and renders the virus non-pathogenic. In addition, in talimogene laherparepvec, the HSV-1 viral gene encoding ICP47 (which blocks viral antigen presentation to major histocompatibility complex class I and II molecules) has been functionally deleted. Functional deletion of ICP47 also leads to earlier expression of US11, a gene that promotes virus growth in tumor cells without decreasing tumor selectivity. Finally, the coding sequence for human GM-CSF, a cytokine involved in the stimulation of immune responses, has been inserted into the viral genome of talimogene laherparepvec. The insertion of the gene encoding human GM-CSF is such that it replaces nearly all of the ICP34.5 gene, ensuring that any potential recombination event between talimogene laherparepvec and wild-type virus could only result in a disabled, non-pathogenic virus and could not result in the generation of wild-type virus carrying the gene for human GM-CSF. The HSV thymidine kinase (TK) gene remains intact in talimogene laherparepvec, which renders the virus sensitive to anti-viral agents such as acyclovir. Therefore, acyclovir can be used to block talimogene laherparepvec replication, if necessary.

In a prior phase 3 clinical trial, intratumoral injection of talimogene laherparepvec into melanoma metastases improved the durable response rate compared with subcutaneous GM-CSF in patients with advanced melanoma (Andtbacka et al. (2015). Talimogene Laherparepvec Improves Durable Response Rate in Patients With Advanced Melanoma. J Clin Oncol 33, 2780-2788). Promising anti-tumor activity was also demonstrated when talimogene laherparepvec was given together with the checkpoint inhibitor ipilimumab, which blocks the cytotoxic T-cell associated-antigen 4 (CTLA-4) (Chesney, J., Collichio, F., Andtbacka, R. H., Puzanov, I., Glaspy, J. A., Milhem, M., Hamid, O., Cranmer, L., Saenger, Y., Ross, M., et al. (2016). Interim safety and efficacy of a randomized (1:1), open-label phase 2 study of talimogene laherparepvec (T) and ipilimumab (I) vs I alone in unresected, stage IIIB-IV melanoma. Ann Oncol 27 (6), 379-400; Puzanov, I., Milhem, M. M., Minor, D., Hamid, O., Li, A., Chen, L., Chastain, M., Gorski, K. S., Anderson, A., Chou, J., et al. (2016). Talimogene Laherparepvec in Combination With Ipilimumab in Previously Untreated, Unresectable Stage IIIB-IV Melanoma. J Clin Oncol 34, 2619-2626).

Talimogene laherparepvec (IMLYGIC®) was approved as a monotherapy treatment for metastatic melanoma in the U.S., European Union, and Australia in 2015. In OPTiM, a multicenter, phase 3 clinical trial that enrolled patients with metastatic melanoma that could not be surgically removed, patients who received talimogene laherparepvec were significantly more likely to experience a durable response compared with patients who received the comparator therapy, GM-CSF. (Andtbacka RHI, et al., J. Clin Oncol., 33:2780-2788 (2015)).

In addition, the safety of ICP34.5-functionally deleted HSVs has been shown in multiple clinical studies (MacKie et al, Lancet 357: 525-526, 2001; Markert et al, Gene Ther 7: 867-874, 2000; Rampling et al, Gene Ther 7:859-866, 2000; Sundaresan et al, J. Virol 74: 3822-3841, 2000; Hunter et al, J Virol August; 73(8): 6319-6326, 1999).

Talimogene laherparepvec produces a direct oncolytic effect by replication of the virus in the tumor, and induction of an anti-tumor immune response enhanced by the local expression of GM-CSF. Intended clinical effects include, but are not limited to, the destruction of injected tumors; the destruction of local, local-regional, and distant uninjected tumors; a reduction in the development of new metastases; a reduction in the rate of overall progression; and prolonged overall survival.

Talimogene laherparepvec has been tested for efficacy in a variety of in vitro (cell line) and in vivo murine tumor models and has been shown to eradicate tumors or substantially inhibit their growth at doses comparable to those used in clinical studies. Non-clinical evaluation has also confirmed that GM-CSF enhances the immune response generated, enhancing both injected and uninjected tumor responses, and that increased surface levels of MHC class I molecules result from the deletion of ICP47. Talimogene laherparepvec has been injected into normal and tumor-bearing mice to assess its safety. In general, the virus has been well tolerated, and doses up to 1×10⁸ PFU/dose have given no indication of any safety concerns. (See, for example, Liu et al., Gene Ther 10: 292-303, 2003).

Clinical studies have been or are being conducted in several advanced tumor types (advanced solid tumors, melanoma, squamous cell cancer of the head and neck, and pancreatic cancer), with over 400 subjects treated with talimogene laherparepvec (see, for example, Hu et al., Clin Can Res 12: 6737-6747, 2006; Harrington et al., J Clin Oncol. 27(15a):abstract 6018, 2009; Kaufman et al., Ann Surgic Oncol. 17: 718-730, 2010; Kaufman and Bines, Future Oncol. 6(6): 941-949, 2010).

“Talimogene laherparepvec/pembrolizumab combination therapy” refers to the use of both talimogene laherparepvec and pembrolizumab, a pembrolizumab variant or an antigen-binding fragment thereof to treat a cancer, e.g., a primary hepatic cancer and/or a secondary hepatic cancer. Administration of talimogene laherparepvec and pembrolizumab, a pembrolizumab variant or an antigen-binding fragment thereof (i.e., talimogene laherparepvec/pembrolizumab combination therapy) may occur simultaneously (i.e., in the same medicament), concurrently (i.e., in separate medicaments administered at the same time in any order), or sequentially in any order, as described further herein.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length.

“Patient” or “subject” refers to any single subject for which therapy is desired or that is participating in a clinical trial, epidemiological study or used as a control, including humans, non-human primates, mammalian veterinary patients such as cattle, horses, dogs, cats and the like, and research animals such as non-human primates, rats, mice, dogs, rabbits and the like.

Pembrolizumab is a humanized monoclonal antibody that binds to and blocks PD-1. Pembrolizumab works by increasing the ability of the body's immune system to help detect and fight tumor cells by blocking the interaction between PD-1 and its ligands, PD-L1 and PD-L2, thereby activating T lymphocytes which may affect both tumor cells and healthy cells.

Pembrolizumab monotherapy is known to treat melanoma, non-small cell lung cancer and squamous cell carcinoma of the head and neck in affected individuals having higher densities of baseline CD8+ T-cell infiltrations, IFNγ gene signature and PD-L1 expression than levels found in non-responsive individuals.

As used herein, “pembrolizumab” refers to a commercially available monoclonal antibody under the proprietary name of KEYTRUDA® (Merck Sharp & Dohme Corp., Whitehouse Station, N.J.), described in WO2016196173 and U.S. Pat. Nos. 8,354,509 and 8,900,587, incorporated herein by reference in their entireties for all purposes, as well as variants and antigen-binding fragments thereof. Pembrolizumab has been approved by the US FDA for the treatment of certain patients having melanoma, non-small cell lung cancer, head and neck squamous cell cancer, classical Hodgkin lymphoma, urothelial carcinoma, microsatellite instability-high cancer, cervical cancer, primary mediastinal B-cell lymphoma, and gastric cancer. Pembrolizumab can be characterized by one or any combination of the heavy chain domain, light chain domain, heavy chain variable domain, light chain variable domain, heavy chain complementarity-determining and light chain complementarity-determining sequences described Infra.

Pembrolizumab can comprise a heavy chain sequence set forth as QVQLVQSGVEVKKPGASVKVSCKASGYTFTNYYMYWVRQAPGQGLEWMGGINPSNGGTN FNEKFKNRVTLTTDSSTTTAYMELKSLQFDDTAVYYCARRDYRFDMGFDYWGQGTTVTVSS ASTKGPSVFPLAPCSRSTSESTAALGCLVKDYFPEPVTVSWNSGALTSGVHTFPAVLQSSGLY SLSSVVTVPSSSLGTKTYTCNVDHKPSNTKVDKRVESKYGPPCPPCPAPEFLGGPSVFLFPPKP KDTLMISRTPEVTCVVVDVSQEDPEVQFNWYVDGVEVHNAKTKPREEQFNSTYRVVSVLTV LHQDWLNGKEYKCKVSNKGLPSSIEKTISKAKGQPREPQVYTLPPSQEEMTKNQVSLTCLVK GFYPSDIAVEWESNGQPENNYKTTPPVLDSDGSFFLYSRLTVDKSRWQEGNVFSCSVMHEAL HNHYTQKSLSLSLGK (SEQ ID NO:1), and a light chain sequence set forth as EIVLTQSPATLSLSPGERATLSCRASKGVSTSGYSYLHWYQQKPGQ APRLLIYLASYLESGVPARFSGSGSGTDFTLTISSLEPEDFAVYYCQHSRDLPLTFGGGTKVEIK RTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO:2).

Pembrolizumab can comprise a heavy chain variable (VH) domain sequence set forth as QVQLVQSGVEVKKPGASVKVSCKAS GYTFTNYYMYWVRQAPGQGLEWMGGINPSNGGTN FNEKFKNRVTLTTDSSTTTAYMELKSLQFDDTAVYYCARRDYRFDMGFDYWGQGTTVTVSS (SEQ ID NO:3), and a light chain variable (VL) domain set forth as EIVLTQSPATLSLSPGERATLSCRASKGVSTSGYSYLHWYQQKPGQAPRLLIYLASYLESGVP ARFSGSGSGTDFTLTISSLEPEDFAVYYCQHSRDLPLTFGGGTKVEIK (SEQ ID NO:4).

Pembrolizumab can comprise the following heavy chain complementarity-determining regions (HCDRs): NYYMY (HCDR1, SEQ ID NO:5); GINPSNGGTNFN (HCDR2, SEQ ID NO:6); and RDYRFDMGFDY (HCDR3, SEQ ID NO:7).

Pembrolizumab can comprise the following light chain complementarity-determining regions (LCDRs): RASKGVSTSGYSYLH (LCDR1, SEQ ID NO:8); LASYLES (LCDR2, SEQ ID NO:9); and QHSRDLPLT (LCDR3, SEQ ID NO:10).

In certain embodiments, pembrolizumab, a pembrolizumab variant or an antigen-binding fragment thereof is provided comprising heavy chain CDRs SEQ ID NOs: 5, 6 and 7 and light chain CDRs of SEQ ID NOs: 8, 9 and 10.

In other embodiments, pembrolizumab, a pembrolizumab variant or an antigen-binding fragment thereof is provided comprising heavy chain and light chain CDR sequences from a VH/VL sequence pair of SEQ ID NO:3 and SEQ ID NO:4.

In still other preferred embodiments, pembrolizumab, a pembrolizumab variant or an antigen-binding fragment thereof is provided comprising a heavy chain variable region comprising SEQ ID NO:3 or a variant thereof and/or a light chain variable region comprising SEQ ID NO:4 or a variant thereof. In other embodiments, the pembrolizumab variant or antigen-binding fragment thereof comprises a heavy chain variable region comprising as sequence with at least 80% sequence homology or identity (e.g., 80%, 85%, 90%, 95%, 98% or 99%) to SEQ ID NO:3 and/or a light chain variable region comprising a sequence with at least 80% sequence homology or identity (e.g., 80%, 85%, 90%, 95%, 98% or 99%) to SEQ ID NO:4.

As used herein, a “variant of a heavy chain variable region sequence” is a sequence that is identical to the reference sequence, except having up to 17 conservative amino acid substitutions in the framework region (i.e., outside of the CDRs), and preferably having fewer than ten, nine, eight, seven, six or five conservative amino acid substitutions in the framework region. As used herein, a “variant of a light chain variable region sequence” is a sequence that is identical to the reference sequence, except having up to five conservative amino acid substitutions in the framework region (i.e., outside of the CDRs), and preferably having fewer than four, three or two conservative amino acid substitutions in the framework region.

In still other embodiments, pembrolizumab, a pembrolizumab variant or an antigen-binding fragment thereof is provided comprising a heavy chain comprising SEQ ID NO:1 or a variant thereof and/or a light chain comprising SEQ ID NO:2 or a variant thereof. In other embodiments, the pembrolizumab variant or antigen-binding fragment thereof comprises a heavy chain comprising a sequence with at least 80% sequence homology or identity (e.g., 80%, 85%, 90%, 95%, 98% or 99%) to SEQ ID NO:1 and/or a light chain comprising a sequence with at least 80% sequence homology or identity (e.g., 80%, 85%, 90%, 95%, 98% or 99%) to SEQ ID NO:2.

As used herein, a “pembrolizumab variant” refers to a monoclonal antibody which comprises heavy chain and light chain sequences that are identical to those of pembrolizumab, except for having up to five conservative amino acid substitutions in the framework region (i.e., outside of the CDRs), and preferably has less than four, three or two conservative amino acid substitution in the framework region, and having up to 17 conservative amino acid substitutions in the framework region (i.e., outside of the CDRs), and preferably has less than ten, nine, eight, seven, six or five conservative amino acid substitutions in the framework region, and preferably has less than four, three or two conservative amino acid substitution in the framework region. In other words, pembrolizumab and a pembrolizumab variant comprise identical CDR sequences, but differ from each other due to having a conservative amino acid substitution at no more than three or six other positions in their full-length light and heavy chain sequences, respectively. A pembrolizumab variant is substantially the same as or better than pembrolizumab with respect to the following properties: binding affinity to PD-1 and neutralizing effect in vivo.

In certain embodiments, biosimilars of pembrolizumab are provided. In certain embodiments, the term “biosimilar” is used in a manner that is consistent with the working definition promulgated by the U.S. Food and Drug Administration, which defines a biosimilar product to be one that is “highly similar” to a reference product (despite minor differences in clinically inactive components). In practice, there can be no clinically meaningful differences between the reference product and the biosimilar product in terms of safety, purity, and potency (Public Health Service (PHS) Act § 262). In certain embodiments, a double-blind, single-dose comparative pharmacokinetic (PK) crossover study is performed to compare pembrolizumab with a candidate biosimilar antibody to determine comparable bioavailability. In other embodiments, the definition of “biosimilar” is consistent with the definition used by a regulatory authority outside of the U.S.

As used herein, the term “reference product,” is used to refer to commercially available pembrolizumab.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Sample” when referring to a tumor or any other biological material referenced herein, means a sample that has been removed from the subject.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Standard of care systemic anti-cancer therapy” refers to medically-accepted diagnostic and treatment processes that a clinician follows for a particular cancer in a particular patient that may include one or more biological therapies (e.g., immunotherapies) and/or one or more cytotoxic chemotherapies that would be readily known to one of skill in the art. As used herein, standard of care systemic anti-cancer therapy excludes talimogene laherparepvec/pembrolizumab combination therapy.

“Tissue Section” refers to a single part or piece of a tissue sample, e.g., a thin slice of tissue cut from a sample of a normal tissue or of a tumor.

“Treat” or “treating” a primary or a secondary hepatic cancer as used herein means to administer pembrolizumab, a pembrolizumab variant or an antigen-binding fragment thereof and talimogene laherparepvec to a subject diagnosed with a primary or a secondary hepatic cancer to achieve at least one positive therapeutic effect, such as for example, reduced number of cancer cells, reduced tumor size, reduced rate of cancer cell infiltration into peripheral organs, or reduced rate of tumor metastasis or tumor growth.

Positive therapeutic effects in cancer can be measured in a number of ways (See, W. A. Weber, J. Null. Med. 50:1S-10S (2009); Eisenhauer et al., supra). In some preferred embodiments, response to pembrolizumab, a pembrolizumab variant and/or an antigen-binding fragment thereof, and/or talimogene laherparepvec is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a primary or a secondary hepatic cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Tumor” as it applies to a subject diagnosed with, or suspected of having, a primary or a secondary hepatic cancer, refers to a malignant or potentially malignant neoplasm or tissue mass of any size. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load,” refers to the total amount of tumor material distributed throughout the body. Tumor burden refers to the total number of cancer cells or the total size of tumor(s) throughout the body, including lymph nodes and bone marrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

### Methods, Uses and Medicaments

In one aspect, the invention relates to a method for treating cancer in an individual comprising administering to the individual a combination therapy which comprises pembrolizumab, a pembrolizumab variant or antigen-binding fragment thereof, and talimogene laherparepvec.

The combination therapy may also comprise one or more additional therapeutic agents. The additional therapeutic agent may be, e.g., a chemotherapeutic agent, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Examples of chemotherapeutic agents include alkylating agents such as thiotepa and cyclosphosphamide; alkyl sulfonates such as busulfan, improsulfan and piposulfan; aziri dines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancrati statin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylormthine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LYI 17018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks and/or are administered to different parts of the body, e.g., one therapeutic agent is administered intratumorally and one therapeutic agent is administered systemically.

In particularly preferred embodiments, talimogene laherparepvec is administered before administration of pembrolizumab, a pembrolizumab variant or an antigen-binding fragment thereof. In other embodiments, talimogene laherparepvec is administered after administration of pembrolizumab, a pembrolizumab variant or an antigen-binding fragment thereof. In other embodiments, talimogene laherparepvec is administered concurrently with pembrolizumab, a pembrolizumab variant or an antigen-binding fragment thereof.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

In certain embodiments, talimogene laherparepvec is administered intratumorally. In certain embodiments, pembrolizumab, a pembrolizumab variant or an antigen-binding fragment thereof is administered parenterally.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is cancer treatment-naive. In other embodiments, the combination therapy is administered to a patient who failed to achieve a sustained response after prior therapy (e.g., after failed or ineffective therapy with a systemic anti-cancer therapy that is not talimogene laherparepvec/pembrolizumab combination therapy), i.e., is cancer treatment-experienced.

A combination therapy of the invention is typically used to treat a tumor that is large enough to be found by palpation or by imaging techniques well known in the art, such as MRI, ultrasound, or CAT scan.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341: 1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343: 1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy. The optimal dose for pembrolizumab in combination with talimogene laherparepvec may be identified by dose escalation or dose de-escalation of one or both of these agents.

The present invention also provides a medicament which comprises pembrolizumab, a pembrolizumab variant and/or an antigen-binding fragment thereof as described above, and a pharmaceutically acceptable excipient for use in combination with talimogene laherparepvec for treating a primary hepatocellular carcinoma, and/or a secondary hepatic cancer such as a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma.

In some embodiments, a medicament comprising pembrolizumab, a pembrolizumab variant and/or an antigen-binding fragment thereof may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention. In some embodiments, a medicament comprising pembrolizumab is provided in a glass vial which contains about 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg), and water for injection, USP. The solution requires dilution for IV infusion.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346: 1692-1698; Liu et al. (1999) J. Neurol. Neurosurg. Psych. 67:451-456; Portielji et al. (20003) Cancer Immunol. Immunother. 52: 133-144.

In certain embodiments that employ pembrolizumab, a pembrolizumab variant and/or an antigen-binding fragment thereof, the dosing regimen will comprise administering pembrolizumab, a pembrolizumab variant and/or an antigen-binding fragment thereof at a dose of 1, 2, 3, 5 or 10 mg/kg at intervals of about 14 days (±2 days) or about 21 days (±2 days) or about 30 days (±2 days) throughout the course of treatment. In a preferred embodiment, pembrolizumab, a pembrolizumab variant or an antigen-binding fragment thereof is used at a dose of 200 mg (fixed) every 3 weeks.

In other embodiments that employ pembrolizumab, a pembrolizumab variant and/or an antigen-binding fragment thereof in the combination therapy, the dosing regimen will comprise administering pembrolizumab, a pembrolizumab variant and/or an antigen-binding fragment thereof at a dose of from about 0.005 mg/kg to about 10 mg/kg, with intra-patient dose escalation. In other escalating dose embodiments, the interval between doses will be progressively shortened, e.g., about 30 days (±3 days) between the first and second dose, about 21 days (±3 days) between the second and third doses. In certain embodiments, the dosing interval will be about 21 days (±3 days), for doses subsequent to the second dose.

In certain embodiments, a subject will be administered a parenteral dosing, e.g., an intravenous (IV) infusion, of a medicament comprising any of pembrolizumab, a pembrolizumab variant and/or an antigen-binding fragment thereof.

In a preferred embodiment of the invention, pembrolizumab, a pembrolizumab variant and/or an antigen-binding fragment thereof is administered in a liquid medicament at a dose selected from the group consisting of 1 mg/kg every two weeks (Q2W) or every 14 days (Q14D), 2 mg/kg Q2W or Q14D, 3 mg/kg Q2W or Q14D, 5 mg/kg Q2W or Q14D, 10 mg Q2W or Q14D, 1 mg/kg every three weeks (Q3W) or every 21 days (Q21D), 2 mg/kg Q3W or Q21D, 3 mg/kg Q3W or Q21D, 5 mg/kg Q3W or Q21D, 10 mg Q3W or Q21D, and flat-dose equivalents of any of these doses, i.e., such as 200 mg Q3W or Q21D.

In some embodiments, pembrolizumab, a pembrolizumab variant and/or an antigen-binding fragment thereof is provided in a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg or about 400 mg.

In certain exemplary embodiments, pembrolizumab, a pembrolizumab variant and/or an antigen-binding fragment thereof is provided in a dosage of about 200 mg. In other exemplary embodiments, pembrolizumab, a pembrolizumab variant and/or an antigen-binding fragment thereof is provided as a liquid medicament which comprises 25 mg/ml pembrolizumab, 7% (w/v) sucrose, 0.02% (w/v) polysorbate 80 in 10 mM histidine buffer pH 5.5.

In some embodiments, the selected dose of pembrolizumab, a pembrolizumab variant and/or an antigen-binding fragment thereof is administered by IV infusion. In one embodiment, the selected dose of pembrolizumab, a pembrolizumab variant and/or an antigen-binding fragment thereof is administered by IV infusion over a time period of between 25 and 40 minutes, or about 30 minutes.

The present invention also provides a medicament which comprises talimogene laherparepvec and a pharmaceutically acceptable excipient for use in combination with pembrolizumab for treating a primary hepatocellular carcinoma, and/or a secondary hepatic cancer such as a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma (including uveal melanoma), non-small cell lung cancer and clear cell renal cell carcinoma. Talimogene laherparepvec may be suspended in a physiological buffer for intratumoral injection.

In certain embodiments, talimogene laherparepvec is provided at a concentration of about 10³ plaque forming units/mL (PFU/mL), about 10⁴ PFU/mL, about 10⁵ PFU/mL, about 10⁶ PFU/mL, about 10⁷ PFU/mL, about 10⁸ PFU/mL, about 10⁹ PFU/mL or about 10¹⁰ PFU/mL. In particular embodiments, talimogene laherparepvec is provided at a concentration of about 10⁶ PFU/mL, about 10⁷ PFU/mL or about 10⁸ PFU/mL.

In certain embodiments, talimogene laherparepvec is administered by intratumoral injection into injectable tumors at a dose of up to about 4.0 mL, of about 5.0 mL, of about 6.0 mL, of about 7.0 mL or of about 8 mL of 10⁶ PFU/mL at day 1 of week 1, followed by a dose of up to about 4.0 mL, of about 5.0 mL, of about 6.0 mL, of about 7.0 mL or of about 8 mL of 10⁷ or 10⁸ PFU/mL at day 1 of weeks 4 and 7, and every 3 weeks (±3 days) thereafter. The recommended volume of talimogene laherparepvec to be injected into the tumor(s) is dependent on the size of the tumor(s) and would be readily apparent to one of ordinary skill in the art based on the disclosure provided herein in view of the knowledge in the art.

All reasonably injectable lesions should be injected with the maximum dosing volume available on an individual dosing occasion. On each treatment day, prioritization of injections is recommended as follows: any new injectable tumor that has appeared since the last injection; by tumor size, beginning with the largest tumor; any previously uninjectable tumor(s) that is now injectable. The compositions may comprise one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. Neutral buffered saline or saline mixed with specific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed.

In some embodiments, the patient is selected for treatment with the combination therapy of the invention if the patient: (1) has histologically or cytologically confirmed BC, CRC, GEC (adenocarcinoma or SCC), melanoma (including uveal melanoma), NSCLC, or RCC with liver metastases; (2) has received at least one prior standard of care systemic anti-cancer therapy for their locally advanced or metastatic disease; and (3) has measurable liver tumors that are suitable for injection.

In other embodiments, the patient is selected for treatment with the combination therapy of the invention if the patient: (1) has HCC with known disease progression; and (2) has measurable liver tumors that are suitable for injection.

The medicaments described herein may be provided as a kit which comprises a first container and a second container and a package insert. The first container contains at least one dose of a medicament comprising a pembrolizumab, a pembrolizumab variant and/or an antigen-binding fragment thereof, and the second container contains at least one dose of talimogene laherparepvec. The kit can optionally comprise a package insert, or label, which includes instructions for treating a patient for cancer using the medicaments. The first and second containers may be comprised of the same or different shapes (e.g., vials, syringes and bottles) and/or materials (e.g., plastic or glass). The kit may further comprise other materials that may be useful in administering the medicaments, such as diluents, filters, IV bags and lines, needles and syringes. In some preferred embodiments of the kit, the instructions state that the medicaments are intended for use in treating a patient having a primary or secondary hepatic cancer.

### Pharmaceutical Compositions

The invention pertains to uses of the above-described agents for prophylactic and/or therapeutic treatments as described Infra. Accordingly, pembrolizumab, the pembrolizumab variant and/or the antigen-binding fragment thereof and/or talimogene laherparepvec of the present invention can be incorporated into pharmaceutical compositions suitable for administration. Such compositions typically comprise pembrolizumab, the pembrolizumab variant and/or the antigen-binding fragment thereof or talimogene laherparepvec and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous, IS, ICV and/or IT administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack or dispenser together with optional instructions for administration.

The pharmaceutical compositions of the present invention may be administered in a number of ways depending upon whether local or systemic treatment is desired and upon the area to be treated. Administration may be intratumoral or parenteral. Parenteral administration includes intravenous drip, subcutaneous, intraperitoneal or intramuscular injection, intrathecal, or intraventricular administration.

and bases, such as sodium citrate, sodium ascorbate, and the like; sodium citrate is preferred.

In one embodiment, unit doses or measured doses of a composition that include pembrolizumab, a pembrolizumab variant and/or antigen-binding fragment thereof or talimogene laherparepvec are dispensed by an implanted device. The device can include a sensor that monitors a parameter within a subject. For example, the device can include a pump, such as an osmotic pump and, optionally, associated electronics.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and are not intended to be limiting. All patents, patent applications and references described herein are incorporated by reference in their entireties for all purposes.

### EXAMPLE

### Example 1. Multicenter, Open-Label Trial to Evaluate the Safety of Intrahepatic Injection of Talimogene Laherparepvec into Liver Tumors in Combination with Systemic Pembrolizumab

A phase 1b/2 trial was designed in patients with primary hepatocellular carcinoma (HCC) or liver metastases (non-HCC) combining intratumoral injection of talimogene laherparepvec with systemic administration of the anti-PD-1 antibody pembrolizumab

### Primary Objectives

The Part 1 Primary objective is to evaluate the maximum tolerated volume and concentration, as assessed by incidence of dose limiting toxicities (DLTs), of intrahepatic injection of talimogene laherparepvec into liver tumors alone and the maximum tolerated concentration of intrahepatic injection of talimogene laherparepvec into liver tumors in combination with systemic intravenous (IV) administration of pembrolizumab, separately, in subjects with liver metastases (non-HCC) and in subjects with primary HCC. The Part 2 Primary objectives are to evaluate the efficacy and safety, as assessed by objective response rate (ORR) and subject incidence of DLT, respectively, of intrahepatic injection of talimogene laherparepvec in combination with systemic IV administration of pembrolizumab, separately, for each non-HCC tumor type (breast adenocarcinoma (BC), colorectal adenocarcinoma (CRC), gastroesophageal cancer (GEC; adenocarcinoma or squamous cell carcinoma (SCC)), melanoma (such a skin or uveal melanoma), non-small cell lung cancer (NSCLC), and clear cell renal cell carcinoma (RCC)) with liver metastases as well as primary HCC.

### Secondary Objectives

The secondary objectives of the study are as follows:

A. Efficacy

Part 1:

To evaluate the efficacy separately by monotherapy versus combination with cohorts combined for non-HCC and HCC tumors as assessed by: ORR, best overall response (BOR), durable response rate (DRR), duration of response (DOR), response in injected and uninjected lesions, disease control rate (DCR), progression-free survival (PFS), and overall survival (OS).

Part 2:

To evaluate the efficacy in individual tumor types in the non-HCC and HCC groups as assessed by: BOR, DRR, DOR, response in injected and uninjected lesions, DCR, PFS, and OS by primary tumor type.

B. Safety (Parts 1 and 2)

To evaluate the safety in each monotherapy and combination cohorts in both groups of Part 1, as assessed by subject incidence of treatment-emergent and treatment related adverse events. To evaluate the safety separately for each tumor type in Part 2, as assessed by subject incidence of treatment emergent and treatment related adverse events. To estimate the incidence of detectable talimogene laherparepvec DNA in blood and urine. To estimate the incidence of clearance of talimogene laherparepvec DNA from blood and urine. To estimate the rate of detection (per sample) and incidence (per subject) of talimogene laherparepvec DNA and virus at the surface of talimogene laherparepvec injection site, the exterior of occlusive dressing, and the oral mucosa. To estimate the incidence of talimogene laherparepvec DNA detection in lesions suspected to be herpetic in origin.

### Primary Endpoint

Part 1:

Subject incidence of DLTs with intrahepatic injection of talimogene laherparepvec into liver tumors alone and in combination with systemic IV administration of pembrolizumab separately in subjects with non-HCC primary tumors and HCC is assessed.

Part 2:

ORR per the modified immune related response criteria simulating Response Evaluation Criteria in Solid Tumors version 1.1 (irRC RECIST) with intrahepatic injection of talimogene laherparepvec into liver tumors in combination with systemic IV administration of pembrolizumab separately by tumor type (BC, CRC, GEC, melanoma, NSCLC, RCC, and HCC) is assessed. Subject incidence of DLTs for each tumor type is assessed.

### Secondary Endpoints

Efficacy (Part 1):

ORR, BOR, DRR, DOR, responses are assessed in injected and in uninjected lesions. DCR, PFS and OS are assessed separately for non-HCC and HCC tumors in subjects receiving talimogene laherparepvec monotherapy or talimogene laherparepvec/pembrolizumab combination therapy.

Efficacy (Part 2):

BOR, DRR, DOR, response in injected and uninjected lesions, DCR, PFS, and OS by primary tumor type (BC, CRC, GEC, melanoma, NSCLC, RCC, and HCC).

Safety (Parts 1 and 2):

The subject incidence of treatment-related and treatment-emergent adverse events are assessed for monotherapy and combination cohorts in both groups in Part 1 and for each tumor type separately in Part 2.

The incidence of detectable talimogene laherparepvec DNA in blood and urine is measured. The incidence of clearance of talimogene laherparepvec DNA from blood and urine is also measured. Subject incidence and sample rate of detection of talimogene laherparepvec DNA and virus are determined at the surface of the injection site, at the exterior of the occlusive dressing, and in the oral mucosa. Subject incidence of talimogene laherparepvec DNA detection in lesions suspected to be herpetic in origin is performed.

### Study Design

This is a phase 1b/2, multicenter, open-label trial to evaluate the safety of talimogene laherparepvec injected intrahepatically into liver tumors with known progression alone and in combination with systemic IV administration of pembrolizumab, in subjects with non-HCC liver metastases from BC, CRC, GEC, melanoma, NSCLC, RCC and subjects with HCC. The study consists of 2 parts and 2 groups, and Part 2 includes 2 stages.

The objective of Part 1 is to evaluate the safety of intrahepatic injection of talimogene laherparepvec into liver tumors alone and in combination with systemically administered pembrolizumab for the non-HCC (Group A) and HCC (Group B) cohorts separately (Table 1). In the monotherapy cohorts (Cohorts 1-4), the safety of administering increasing concentration (10⁷ plaque forming units (PFU)/mL or 10⁸ PFU/mL) and volumes (maximum up to 4 mL or 8 mL) of talimogene laherparepvec will be determined using a standard 3+3 design. The initial concentration of talimogene laherparepvec in all cohorts is always 10⁶ PFU/mL. Cohort 1 of Group B will be initiated only after safety has been established in Cohort 1 of Group A.

Combination cohorts (Cohorts 5 and 6) in Part 1 will determine the safety of administering intrahepatic injection of talimogene laherparepvec at 10⁷ PFU/mL or 10⁸ PFU/mL sequentially (volume up to 4 mL for both doses) in combination with systemic IV administration of pembrolizumab (200 mg) (Table 1). A modified toxicity probability interval (mTPI) design will be used to determine safety in the combination cohorts.

TABLE 1
Summary of Part 1 cohorts.

| | | | Second and | |
|---|---|---|---|---|
| | | Initial Talimogene | Subsequent Talimogene | |
| | Talimogene | Laherparepvec | Laherparepvec | |
| Cohorts | laherparepvec | Concentration | Concentration | Pembrolizumab |
| in Part 1 | Volume (mL) | (PFU/mL) | (PFU/mL) | dose (mg) |
| 1 | 4 | 10⁶ | 10⁷ | n/a |
| 2 | 4 | 10⁶ | 10⁸ | n/a |
| 3 | 8 | 10⁶ | 10⁸ | n/a |
| 4^{a} | 8 | 10⁶ | 10⁷ | n/a |
| 5 | 4 | 10⁶ | 10⁷ | 200 |
| 6 | 4 | 10⁶ | 10⁸ | 200 |

DLT: dose-limiting toxicity;
n/a = not applicable;
PFU: plaque forming unit.
^{a}Cohort will be opened only if one of these conditions are met: 1) DLT ≥ 33% in Cohort 2; 2) DLT ≥ 33% in Cohort 3 and Part 2 dose for talimogene laherparepvec not determined yet; or 3) DLT ≥ 33% in Cohort 3 and Part 2 concentration for talimogene laherparepvec is determined to be 10⁷ PFU/mL.

Once the maximum tolerated concentration (MTC) is determined from cohorts 5 and 6, Part 2 will open to evaluate efficacy and safety of the combination treatment in the respective tumor types (six tumor non-HCC types in Group A and HCC tumor type in Group B) with the maximum tolerated concentration (MTC) of talimogene laherparepvec, as determined from Cohorts 5 and 6, in combination with systemic pembrolizumab Timing of opening Part 2 for the non-HCC (Group A) and HCC (Group B) will be based on when the MTC for talimogene laherparepvec (combination cohorts) is determined for the respective Groups. Talimogene laherparepvec will be used at a maximum volume of 4 mL (or up to 8 mL if safety of administering 8 mL is established from monotherapy cohorts 3 and 4 at the time of opening Part 2). If the safety of administering up to 8 mL from the monotherapy cohorts is not established at the time of opening Part 2, newly enrolled subjects in Part 2 will be allowed to receive up to a maximum of 8 mL, if and when, safety of administering 8 mL in monotherapy is established.

Part 2 consists of 2 stage design to evaluate the efficacy and safety of talimogene laherparepvec in combination with systemic pembrolizumab Efficacy and safety will be evaluated in each of the six non-HCC tumor types from Group A separately. Similarly, the efficacy and safety of the combination treatment will be determined for Group B HCC subjects. The primary analysis of efficacy will include subjects that receive any volume of talimogene laherparepvec.

Sample Size:

The total number of subjects who will participate in Part 1 and Part 2 of the study is anticipated to be approximately 3 to 244 subjects (approximately 3 to 104 subjects in Part 1; approximately 70 to 147 subjects in Part 2). This estimate includes approximately 147 Full Analysis Set subjects that receive the combination treatment in Parts 1 and 2, and up to 48 DLT evaluable subjects from Part 1 monotherapy cohorts. The Part 2 efficacy will include all treated subjects; therefore, dose limiting toxicity non-evaluable subjects will not be replaced in Part 2.

### Summary of Subject Eligibility Criteria

Key Inclusion Criteria:

Subjects must be age≥18 years at the time of informed consent. They must have histologically or cytologically confirmed BC, CRC, GEC, melanoma, NSCLC, or RCC with liver metastases or HCC. Non-HCC subjects must have received at least 1 prior standard of care systemic anti-cancer therapy for their locally advanced or metastatic disease. For the combination cohorts (Cohorts 5 and 6 in Part 1) and Part 2, subjects with metastatic melanoma or NSCLC do not need to have received prior therapy. Subjects must have measurable liver tumors that are suitable for injection. Eastern Cooperative Oncology Group (ECOG) performance status must be 0 or 1, and life expectancy should be approximately 5 months or more. Adequate hematological, renal, hepatic, and coagulation function is required. Liver function tests may be mildly abnormal but within the parameters defined in Section 4.1.1. Child Pugh score must be A to B7.

Key Exclusion Criteria:

Subjects must not be candidates for hepatic surgery or locoregional therapy of liver tumors with curative intent or planned systemic anti-cancer therapy. Liver tumors must not be estimated to invade approximately more than one-third of the liver. Liver tumor-directed therapy, hepatic surgery, antibody-based therapy, or immunotherapy must not have been performed<28 days, chemotherapy<21 days, and targeted small molecule therapy or hormonal therapy<14 days prior to enrollment. Subjects must have either no central nervous system (CNS) metastasis or carcinomatous meningitis, or if CNS metastasis is present, must have stable treated cerebral metastases from BC, NSCLC, RCC, CRC, or melanoma. Subjects must not have symptomatic auto-immune disease or be immunosuppressed. They must not have a history of solid organ transplantation. For non-HCC, there must not be acute or chronic hepatitis B virus (HBV) or hepatitis C virus (HCV) infection. For HCC with prior hepatitis B and/or C infection, HBV and/or HCV viral load by real time polymerase chain reaction (qPCR) must be undetectable, and they must not have had recent treatment within 12 weeks for HBV or HCV with certain antiviral medications. There should be no macroscopic intravascular invasion of tumors into the main portal vein, hepatic vein, or vena cava. Subjects must not: have active herpetic skin lesions or prior complications of herpetic infection (e.g., herpetic keratitis or encephalitis); require treatment with an anti-herpetic drug; have received live-virus vaccination within 30 days of planned treatment start; have previous therapy with talimogene laherparepvec, oncolytic viruses, or tumor vaccine. They must not require concomitant treatment with warfarin. Subjects in the combination treatment cohort must not have: a history or evidence of psychiatric, substance abuse, or any other clinically significant disorder; toxic effects of the most recent prior chemotherapy not resolved to grade 1 or less (except alopecia); received a transfusion of blood products within 28 days prior to study enrollment; or expected other cancer therapy while on study with the exception of local radiation to the site of bone or other metastasis for palliative treatment. Male subjects of reproductive potential in the combination treatment must be willing to use acceptable methods of effective contraception during treatment and through 4 months after the last dose of pembrolizumab

### Investigational Product

Talimogene laherparepvec will be administered by image guided injection (either by ultrasound (US) or computed tomography (CT) scan) into injectable liver lesions only. Dosing concentrations and volumes will be determined during dose escalation in Part 1. The initial concentration for all subjects will be at 10⁶ PFU/mL. Twenty-one (+3) days later, either 10⁷ or 10⁸ PFU/mL will be given. Subsequent doses will be given every 21 days (Q21D; ±3 days) thereafter. The volume will be either up to 4 mL or 8 mL. Up to 6 doses of talimogene laherparepvec may be administered (including 10⁶ PFU/mL dose), and there is an investigator option to continue for up to 6 additional doses Q21D (±3 days). Talimogene laherparepvec will be provided at either 10⁶ PFU/mL or 10⁸ PFU/mL concentrations.

### Pembrolizumab

Pembrolizumab at a dose of 200 mg will be administered every 3 weeks (Q3W; ±3 days) using 30 minute IV infusion. When talimogene laherparepvec and pembrolizumab are administered on the same day, talimogene laherparepvec should be administered first, if possible.

Subjects can receive up to 35 cycles (approximately 24 months) with pembrolizumab During that time, subjects may continue until progressive disease (PD) per the modified irRC-RECIST, unacceptable toxicity, withdrawal of consent, physician's decision to stop therapy for the subject, or sponsor's decision to terminate the study. Discontinuation of treatment may be considered for subjects who have attained a confirmed complete response (CR) that have been treated for at least 8 cycles (24 weeks) with pembrolizumab, and had at least 2 cycles of pembrolizumab beyond the date when the initial CR was declared.

### Procedures

Disease-Specific Assessments:

The following disease specific assessments are performed: (1) liver tumor biopsy unless already performed as standard of care; (2) radiographic tumor assessment; (3) documentation of concomitant medications; and (4) review of adverse events, disease related events, and serious adverse events.

What is claimed is:

1. A method of treating a cancer in a subject, said method comprising administering to said subject:
talimogene laherparepvec; and
pembrolizumab, a pembrolizumab variant or an antigen-binding fragment thereof,
wherein said cancer is selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

2. A method of treating a primary or a secondary hepatic cancer in a subject comprising administering to said subject:
talimogene laherparepvec; and
pembrolizumab, a pembrolizumab variant or an antigen-binding fragment thereof.

3. The method of claim 2, wherein the primary hepatic cancer is a primary hepatocellular carcinoma (HCC).

4. The method of claim 2, wherein the secondary hepatic cancer is a metastasis of a cancer selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

5. The method of any one of claims 1-4, wherein talimogene laherparepvec is administered to the subject intratumorally.

6. The method of any one of claims 1-4, wherein pembrolizumab, the pembrolizumab variant or the antigen-binding fragment thereof is administered to the subject systemically.

7. The method of any one of claims 1-4, wherein talimogene laherparepvec is administered to the subject prior to the administration of pembrolizumab, the pembrolizumab variant or the antigen-binding fragment thereof.

8. The method of any one of claims 1-4, wherein a reduction in size of the injected tumor occurs after administering talimogene laherparepvec and pembrolizumab, the pembrolizumab variant or the antigen-binding fragment thereof.

9. The method of any one of claims 1-4, wherein talimogene laherparepvec is administered sequentially as an initial dose followed by one or more secondary doses.

10. The method of any one of claims 1-4, wherein pembrolizumab, the pembrolizumab variant or the antigen-binding fragment thereof is administered sequentially as an initial dose followed by one or more secondary doses.

11. The method of any one of claims 1-4, wherein talimogene laherparepvec is administered sequentially as an initial dose followed by one or more secondary doses, and wherein pembrolizumab, the pembrolizumab variant or the antigen-binding fragment thereof is administered sequentially and concomitantly with one or more secondary doses of talimogene laherparepvec.

12. The method of claim 11, wherein talimogene laherparepvec is administered intratumorally and wherein pembrolizumab, the pembrolizumab variant or the antigen-binding fragment thereof is administered systemically.

13. The method of claim 11, wherein talimogene laherparepvec and pembrolizumab, the pembrolizumab variant or the antigen-binding fragment thereof are administered intratumorally.

14. A method of treating a cancer in a subject that is poorly responsive to standard of care systemic anti-cancer therapy, said method comprising administering to said subject:
talimogene laherparepvec; and
pembrolizumab, a pembrolizumab variant or an antigen-binding fragment thereof,
wherein the standard of care systemic anti-cancer therapy does not comprise talimogene laherparepvec/pembrolizumab combination therapy, and
wherein said cancer is selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

15. A method of treating a primary or a secondary hepatic cancer in a subject that is poorly responsive to standard of care systemic anti-cancer therapy, said method comprising administering to said subject:
talimogene laherparepvec; and
pembrolizumab, a pembrolizumab variant or an antigen-binding fragment thereof,
wherein the standard of care systemic anti-cancer therapy does not comprise talimogene laherparepvec/pembrolizumab combination therapy.

16. The method of claim 15, wherein the primary hepatic cancer is a primary hepatocellular carcinoma.

17. The method of claim 15, wherein the secondary hepatic cancer is a metastasis of a cancer selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

18. The method of any one of claims 14-17, wherein talimogene laherparepvec is administered to the subject intratumorally.

19. The method of any one of claims 14-17, wherein pembrolizumab, the pembrolizumab variant, or the antigen-binding fragment thereof is administered to the subject systemically.

20. A method of treating a cancer in a subject that progressed during standard of care systemic anti-cancer therapy, said method comprising administering to said subject:
talimogene laherparepvec; and
pembrolizumab, a pembrolizumab variant or an antigen-binding fragment thereof,
wherein the standard of care systemic anti-cancer therapy does not comprise talimogene laherparepvec/pembrolizumab combination therapy, and
wherein said cancer is selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

21. A method of treating a primary or a secondary hepatic cancer in a subject that progressed during standard of care systemic anti-cancer therapy, said method comprising administering to said subject:
talimogene laherparepvec; and
pembrolizumab, a pembrolizumab variant or an antigen-binding fragment thereof,
wherein the standard of care systemic anti-cancer therapy does not comprise talimogene laherparepvec/pembrolizumab combination therapy.

22. The method of claim 21, wherein the primary hepatic cancer is a primary hepatocellular carcinoma.

23. The method of claim 21, wherein the secondary hepatic cancer is a metastasis of a cancer selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

24. The method of any one of claims 20-23, wherein talimogene laherparepvec is administered to the subject intratumorally.

25. The method of any one of claims 20-23, wherein pembrolizumab, the pembrolizumab variant, or the antigen-binding fragment thereof is administered to the subject systemically.

26. A method of treating a cancer in a subject that is resistant to standard of care systemic anti-cancer therapy, said method comprising administering to said subject:
talimogene laherparepvec; and
pembrolizumab, a pembrolizumab variant or an antigen-binding fragment thereof,
wherein standard of care systemic anti-cancer therapy does not comprise talimogene laherparepvec/pembrolizumab combination therapy, and
wherein said cancer is selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

27. A method of treating a primary or a secondary hepatic cancer in a subject that is resistant to standard of care systemic anti-cancer therapy, said method comprising administering to said subject:
talimogene laherparepvec; and
pembrolizumab, a pembrolizumab variant or an antigen-binding fragment thereof,
wherein standard of care systemic anti-cancer therapy does not comprise talimogene laherparepvec/pembrolizumab combination therapy.

28. The method of claim 27, wherein the primary hepatic cancer is a primary hepatocellular carcinoma.

29. The method of claim 27, wherein the secondary hepatic cancer is a metastasis of a cancer selected from the group consisting of: hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

30. The method of any one of claims 26-29, wherein talimogene laherparepvec is administered to the subject intratumorally.

31. The method of any one of claims 26-29, wherein pembrolizumab, the pembrolizumab variant, or the antigen-binding fragment thereof is administered to the subject systemically.

32. A method of treating a cancer in a subject, said method comprising administering to said subject:
talimogene laherparepvec intratumorally as an initial dose followed by one or more secondary doses; and
pembrolizumab, a pembrolizumab variant or an antigen-binding fragment thereof systemically as an initial dose followed by one or more secondary doses.

33. A method of treating a primary or a secondary hepatic cancer in a subject, said method comprising administering to said subject:
talimogene laherparepvec intratumorally as an initial dose followed by one or more secondary doses; and
pembrolizumab, a pembrolizumab variant or an antigen-binding fragment thereof systemically as an initial dose followed by one or more secondary doses.

34. The method of claim 32 or 33, wherein the secondary doses are administered every three weeks (Q3W).

35. The method of claim 32 or 33, wherein the initial dose of talimogene laherparepvec is administered on day 1 of week 1 and a secondary dose of talimogene laherparepvec is administered on day 1 of week 4, on day 1 of week 7, and Q3W thereafter.

36. The method of claim 35, wherein the initial dose of pembrolizumab, pembrolizumab variant or antigen-binding fragment thereof is administered on day 1 of week 4, and a secondary dose of pembrolizumab, pembrolizumab variant or antigen-binding fragment thereof is administered on day 1 of week 7, and Q3W thereafter.

37. The method of claim 36, wherein the initial dose of talimogene laherparepvec is administered at a dose of 10⁶ plaque forming units (PFU)/mL and the secondary doses of talimogene laherparepvec are administered at a dose of 10⁷ or 10⁸ PFU/mL.

38. The method of claim 37, wherein the initial dose and the secondary doses are up to about 4 mL or about 8 mL.

39. The method of claim 38, wherein the initial dose and/or the secondary doses are each up to about 4 mL.

40. The method of claim 38, wherein the initial dose and/or the secondary doses are each up to about 8 mL.

41. The method of claim 36, wherein the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.

42. Talimogene laherparepvec for use in treating a cancer in a subject in combination with pembrolizumab, a pembrolizumab variant or an antigen-binding fragment thereof, wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

43. Pembrolizumab, a pembrolizumab variant or an antigen-binding fragment thereof for use in treating a cancer in a subject in combination with talimogene laherparepvec, wherein said cancer is selected from the group consisting of hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

44. Talimogene laherparepvec for use in treating a primary or a secondary hepatic cancer in a subject in combination with pembrolizumab, a pembrolizumab variant or an antigen-binding fragment thereof.

45. Pembrolizumab, a pembrolizumab variant or an antigen-binding fragment thereof for use in treating a primary or a secondary hepatic cancer in a subject in combination with talimogene laherparepvec.

46. The use of claim 44 or 45, wherein the primary hepatic cancer is an HCC.

47. The use of claim 44 or 45, wherein the secondary hepatic cancer is a metastasis of a cancer selected from the group consisting of hepatocellular carcinoma, breast adenocarcinoma, colorectal adenocarcinoma, gastroesophageal adenocarcinoma, gastroesophageal squamous cell carcinoma, melanoma, non-small cell lung cancer and clear cell renal cell carcinoma.

48. The use of any one of claims 42-47, wherein talimogene laherparepvec is administered to the subject intratumorally.

49. The use of any one of claims 42-47, wherein pembrolizumab, the pembrolizumab variant or the antigen-binding fragment thereof is administered to the subject systemically.

50. The use of any one of claims 42-47, wherein talimogene laherparepvec is administered to the subject prior to the administration of pembrolizumab, the pembrolizumab variant or the antigen-binding fragment thereof.

51. The use of any one of claims 42-47, wherein a reduction in size of the injected tumor occurs after administering talimogene laherparepvec and pembrolizumab, the pembrolizumab variant or the antigen-binding fragment thereof.

52. The use of any one of claims 42-47, wherein talimogene laherparepvec is administered sequentially as an initial dose followed by one or more secondary doses.

53. The use of any one of claims 42-47, wherein pembrolizumab, the pembrolizumab variant or the antigen-binding fragment thereof is administered sequentially as an initial dose followed by one or more secondary doses.

54. The use of any one of claims 42-47, wherein talimogene laherparepvec is administered sequentially as an initial dose followed by one or more secondary doses, and wherein pembrolizumab, the pembrolizumab variant or the antigen-binding fragment thereof is administered sequentially and concomitantly with one or more secondary doses of talimogene laherparepvec.

55. The use of claim 54, wherein talimogene laherparepvec is administered intratumorally and wherein pembrolizumab, the pembrolizumab variant or the antigen-binding fragment thereof is administered systemically.

56. The use of claim 54, wherein talimogene laherparepvec and pembrolizumab, the pembrolizumab variant or the antigen-binding fragment thereof are administered intratumorally.

57. The use of claim 55 or 56, wherein the secondary doses are administered Q3W.

58. The use of claim 55 or 56, wherein the initial dose of talimogene laherparepvec is administered on day 1 of week 1 and a secondary dose of talimogene laherparepvec is administered on day 1 of week 4, on day 1 of week 7, and Q3W thereafter.

59. The use of claim 58, wherein the initial dose of pembrolizumab, pembrolizumab variant or antigen-binding fragment thereof is administered on day 1 of week 4 and a secondary dose of pembrolizumab, pembrolizumab variant or antigen-binding fragment thereof is administered on day 1 of week 7 and Q3W thereafter.

60. The use of claim 59, wherein the initial dose of talimogene laherparepvec is administered at a dose of 10⁶ PFU/mL and the secondary doses of talimogene laherparepvec are administered at a dose of 10⁷ or 10⁸ PFU/mL.

61. The use of claim 60, wherein the initial dose and the secondary doses are up to about 4 mL or about 8 mL.

62. The use of claim 61, wherein the initial dose and/or the secondary doses are each up to about 4 mL.

63. The use of claim 61, wherein the initial dose and/or the secondary doses are each up to about 8 mL.

64. The use of claim 59, wherein the initial dose of pembrolizumab, pembrolizumab variant or antigen-binding fragment thereof is administered at a dose of about 200 mg and the secondary doses of pembrolizumab, pembrolizumab variant or antigen-binding fragment thereof are administered at a dose of about 200 mg.


SEQUENCE LISTING (10 sequences)

SEQ ID NO: 1 - protein, 447 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, Artificial Sequence [sequence omitted]

SEQ ID NO: 4 - protein, 111 aa, Artificial Sequence [sequence omitted]

SEQ ID NO: 5 - protein, 5 aa, Artificial Sequence
  source: /note=“Description of Artificial Sequence Synthetic peptide”
NYYMY

SEQ ID NO: 6 - protein, 12 aa, Artificial Sequence
  source: /note=“Description of Artificial Sequence Synthetic peptide”
GINPSNGGTNFN

SEQ ID NO: 7 - protein, 11 aa, Artificial Sequence
  source: /note=“Description of Artificial Sequence Synthetic peptide”
RDYRFDMGFDY

SEQ ID NO: 8 - protein, 15 aa, Artificial Sequence
  source: /note=“Description of Artificial Sequence Synthetic peptide”
RASKGVSTSGYSYLH

SEQ ID NO: 9 - protein, 7 aa, Artificial Sequence
  source: /note=“Description of Artificial Sequence Synthetic peptide”
LASYLES

SEQ ID NO: 10 - protein, 9 aa, Artificial Sequence
  source: /note=“Description of Artificial Sequence Synthetic peptide”
QHSRDLPLT